(19) United States
(12) Patent Application Publication
(10) Pub. No.: US 2003/0109507 A1
(43) Pub. Date: Jun. 12, 2003

(54) Medicaments based on progestins for dermal use

(75) Inventors: Christian Franke, Allensbach (DE); Karsten Beckmann, Stockach (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DR.KADE Pharmazeutische Fabrik GmbH, Berlin (DE)

(21) Appl. No.: 10/247,629

(22) Filed: Sep. 20, 2002

(30) Foreign Application Priority Data
Sep. 21, 2001 (DE) 101 46 541.6

(51) Int. Cl. A61K 31/56; A61K 31/375

(52) U.S. Cl. 514/171; 514/474; 424/449

(57) ABSTRACT

The present invention relates to semisolid transcutaneous medicaments based on at least one oxidation-sensitive progestin or a pharmaceutically acceptable derivative thereof. The medicaments comprise ascorbic acid, an ascorbic acid derivative or a salt thereof and have excellent stability. Corresponding gels which comprise a combination of norethisterone acetate and estradiol are described in particular.

The present invention relates to medicaments based on progestins for dermal use, for example in the form of a gel. In particular, combination products based on norethisterone acetate and estrogens, for example estradiol, are described. The medicaments are used in particular in hormone replacement therapy for peri- or postmenopausal women.

A decline in the estradiol production by the ovaries during and after the menopause or following ovarectomy leads to a wide variety of symptoms. These symptoms are normally treated by estrogen replacement and, on long-term use of estrogen-containing products during the menopause in non-hysterectomized women, additional administration, sequentially or continuously, of a progestin ought to take place. Corresponding combination products for oral therapy are available (EP-A 0 136 011).

U.S. Pat. No. 5,955,454 and DE-A 199 25 290 respectively describe progestogen-containing and estrogen- or progestin-containing medicaments which can be administered nasally.

Available for transcutaneous therapy are, in particular, active ingredient-containing plasters (cf., for example, EP-A 0 573 133 and GB-A 2 208 147). One disadvantage of the plasters is their relatively poor local tolerability. Thus, so-called plaster allergies are common among some users. In this regard semisolid preparations such as gels have distinct advantages.

For example, EP-A 0 811 381 describes a gel for transcutaneous administration of estrogens, progestins and mixtures thereof. A combination of lauryl alcohol and diethylene glycol monoethyl ether is proposed in order to achieve an appropriate transdermal permeation of estradiol and norethisterone acetate. WO 90/11064 discloses the use of certain esters such as propylene glycol monolaurate, methyl laurate and the like in place of lauryl alcohol. Gels for topical application of 19-norprogestones are described in WO 99/48477.

However, such semisolid drug forms are associated, for some of the normally used sex hormones and, in particular, for norethisterone and its derivatives and other progestins of related structure, with the problems of oxidative decomposition of the active ingredients, which has no practical importance in solid drug forms (for example DE-A 44 12 464). Formulation of semisolid, norethisterone-containing preparations complying with the requirements for pharmaceutical products has therefore to date given only unsatisfactory results, in contrast to solid products such as tablets.

The object on which the present invention is based, to formulate oxidation-sensitive progestins and, in particular, norethisterone or norethisterone derivatives as semisolid drug form with the required medicament stability, is achieved by the present invention through the addition of ascorbic acid and ascorbic acid derivatives. According to a further aspect, it was also an object to indicate well-tolerated formulations, that is to say, for example, those which contain minimal amounts of lower alcohols, in particular little ethanol.

The present invention therefore relates to semisolid transcutaneous medicaments based on at least one oxidation-sensitive progestin or a pharmaceutically acceptable derivative thereof, which comprise ascorbic acid, a pharmaceutically acceptable derivative and/or salt thereof.

The proportion of ascorbic acid and derivatives thereof in the medicaments of the invention is also referred to as the ascorbic acid component and may correspond to single substances but also a mixture of two or more different ones.

The ascorbic acid or ascorbic acid derivatives used according to the invention are characterised by their antioxidant and, in particular, stabilizing effect for oxygen-sensitive substances and can be incorporated into semisolid medicaments.

The term “ascorbic acid” represents 5-[1,2-dihydroxyethyl]-3,4-dihydroxy-5H-furan-2-one of the formula (I)

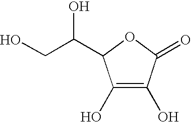

The ascorbic acid derivatives include, in particular ascorbic esters, e.g. esters of the formula (II)

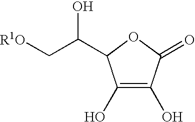

in which R¹ is an aliphatic or aromatic radical having 1 to 30 carbon atoms. Ascorbyl laurate, myristate, palmitate, oleate and stearate may be mentioned as examples. Preferred esters are ascorbyl palmitate and ascorbyl stearate.

It is also possible to use ascorbyl-2-phosphates.

The salts of these compounds, i.e. of ascorbic acid and derivatives thereof, include, in particular, the corresponding base addition salts.

The base addition salts include salts with inorganic bases, for example metal hydroxides or carbonates of alkali metals, alkaline earth metals or transition metals, or with organic bases, for example basic amino acids such as arginine and lysine, ammonia, amines, e.g. methylamine, dimethylamine, trimethylamine, triethylamine, ethylamine, diethylamine, ethylenediamine, ethanolamine, diethanolamine, triethanolamine, 1-amino-2-propanol, 3-amino-1-propanol or hexamethylenetetramine, saturated cyclic amines having 4 to 6 ring carbon atoms, such as piperidine, piperazine, pyrrolidine and morpholine, and other organic bases, for example N-methylglucamine, creatine and tromethamine, and quaternary ammonium compounds such as the ammonium ion, tetramethylammonium ion and the like.

Preferred salts are formed with inorganic bases such as, for example, Na, K, Mg, Ca, Zn, Cr and Fe salts, and salts with the ammonium ion or quaternary ammonium compounds.

Ascorbic acid forms readily soluble in water are preferred, that is to say in particular ascorbic acid itself and salts thereof.

The representation chosen here for compounds of the formulae (I) and (II) includes isomeric forms of these compounds. Particular mention may be made of geometric and stereoisomers such as cis/trans isomers, enantiomers or diastereoisomers, and tautomers, which in the present case are attributable in particular to the enol structure. Besides the essentially pure isomers, the compounds of the formula (I) also include mixtures of isomers thereof, e.g. mixtures of stereoisomers. Thus, besides the preferred L-isomers, mention should also be made for example of isoascorbic acid and isoascorbic acid derivatives. The L-isomers are preferred.

Medicaments of the invention comprise sufficient ascorbic acid to ensure the required medicament stability. In relation to the active ingredient content, stability means for the purposes of the invention a decrease in the content of progestin and in particular of norethisterone of less than 10% by weight and preferably of less than 5% by weight, in each case based on the original amount of active ingredient, during a period of 36 months at room temperature (about 25° C.). According to a particular aspect it was moreover intended that the content of oxidative decomposition products, such as the content of 6-hydroxy or 6-keto derivatives, e.g. 6α- and 6β-hydroxynorethisterone and 6-ketonorethisterone, or corresponding derivatives thereof, be less than about 2% by weight, preferably less than 1% by weight, and in particular less than 0.5% by weight, in each case based on the original active ingredient content.

Medicaments of the invention ordinarily comprise from 0.01 to 1.5% by weight, and preferably 0.1 to 1% by weight, of ascorbic acid, for example about 0.2% by weight of L-ascorbic acid. Ascorbic acid derivatives and salts are employed in corresponding amounts.

In a particular embodiment, medicaments of the invention comprise at least one further antioxidant in addition to ascorbic acid, ascorbic acid salts or ascorbic acid derivatives.

These can be selected in particular from amino acids (e.g. glycine, histidine, lysine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated dosages (e.g. pmol to mmol/kg), α,β-unsaturated caboxylic acids (e.g. fumaric acid), α-hydroxy acids (e.g. citric acid, lactic acid, malic acid), humic acid, bile acid, bile extracts, bilirubin, biliverdin, EDTA, EGTA and derivatives thereof, e.g. propyl gallate, unsaturated fatty acids and derivatives thereof (e.g. γ-linolenic acid, linoleic acid, oleic acid), folic acid and derivatives thereof, ubiquinone and ubiquinol and derivatives thereof, tocopherols and derivatives (e.g. vitamin E acetate), vitamin A and derivatives (vitamin A palmitate) and coniferyl benzoate from gum benzoin, rutic acid and derivatives thereof, butylated hydroxytoluene, butylated hydroxyanisole, nordihydroguaiaretic acid, trihydroxybutyrophenone, uric acid and derivatives thereof, mannose and derivatives thereof, sesamol, sesamolin, stilbenes and derivatives thereof (e.g. stilbene oxide, trans-stilbene oxide) and the derivatives suitable according to the invention (salts, esters, ethers, sugars, nucleotides, nucleosides, peptides and lipids) of these antioxidants mentioned.

Preference is given according to the invention to the combination of ascorbic acid, ascorbic acid salts or ascorbic acid derivatives with at least one chelating agent.

Preferred chelating agents are able to complex heavy metal ions. Particular mention should be made here of amino polycarboxylic acids, for example, ethylenediaminetetraacetic acid (EDTA), diethylenetriaminepentaacetic acid (DTPA), N-hydroxyethylethylenediaminetriacetic acid (HEDTA), nitrilotriacetic acid and salts thereof. EDTA or a pharmaceutically acceptable salt, for example the disodium salt, is preferably used.

Where present, medicaments of the invention ordinarily comprise from 0.01 to 1% by weight and preferably 0.05 to 0.5% by weight of at least one chelating agent, for example about 0.1% by weight of EDTA 2Na.

Medicaments of the invention may, in particular, also comprise α-tocopherol or α-tocopherol derivatives and/or, in particular, fumaric acid. Further additions usable in combination with ascorbic acid, ascorbic acid salts or ascorbic acid derivatives are also amino acids, especially lysine, and/or magnesium sulfate or aluminum sulfate.

The term “α-tocopherol” refers according to the invention to 2-[4,8,12-trimethyltridecyl]-3,4-dihydro-2H-1-benzpyran-6-ole of the formula (III)

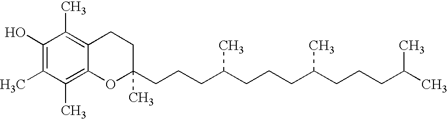

which is also referred to as vitamin E.

The α-tocopherol derivatives include, in particular, α-tocopherol esters, e.g. esters of the formula (IV)

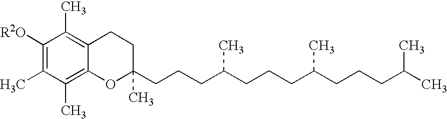

in which R² is an aliphatic or aromatic radical having 1 to 30 carbon atoms. α-Tocopherol fatty acid esters such as linoleic, oleic, linolenic, palmitic, myristic and stearic acid esters, α-tocopherol acetate, α-tocopherol hydrogen succinate and α-tocopherol phosphate should be mentioned here as examples.

Where present, medicaments of the invention ordinarily comprise from 0.01 to 1.5% by weight, and preferably 0.05 to 1% by weight, of at least one other antioxidant, in particular about 0.1% by weight of lysine and/or fumaric acid.

Oxidation-sensitive progestins include, in particular, progestins with a basic steroid framework which is unsaturated in the 4,5 position, of the formula (V)

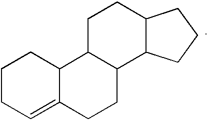

These are, in particular,

Allylestrenol (17α-allyl-4-estren-17-ol) of the formula (Va)

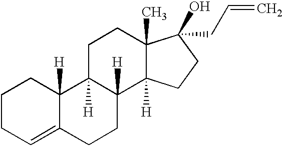

Desogestrel (13-ethyl-11-methylene-18,19-dinor-17α-pregn-4-en-20-yn-17-ol) of the formula (Vb)

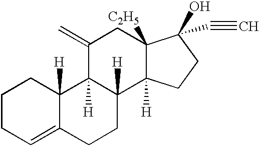

Gestodene (13-ethyl-17β-hydroxy-18,19-dinor-4, 15-pregnadien-20-yn-3-one) of the formula (Vc)

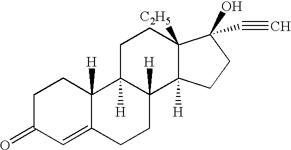

Hydroxyprogesterone (17α-hydroxypregn-4-ene-3,20-dione) of the formula (Vd)

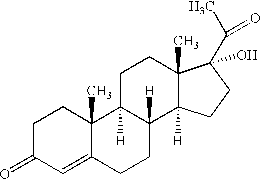

Lynestrenol (19-nor-17α-pregn-4-en-20-yn-17-ol) of the formula (Ve)

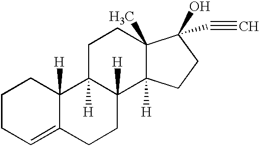

Norgestrel ((±)-13-ethyl-17-hydroxy-18,19-dinor-17α-pregn-4-en-20-yn-3-one) und levonorgestrel ((−)-13-ethyl-17-hydroxy-18,19-dinor-17α-pregn-4-en-20-yn-3-one) of the formula (Vf)

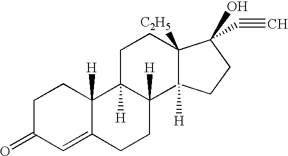

Norethisterone (17-hydroxy-19-nor-17α-pregn-4-en-20-yn-3-one) of the formula (Vg)

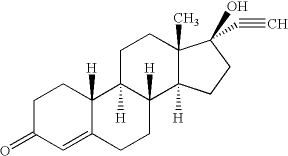

which is also referred to as 17α-ethynyl-19-nortestosterone, norethindrone or norpregneminolone is preferred according to the invention.

The progestin derivatives which can be used according to the invention include, in particular, the esters thereof which can be administered transcutaneously. These include in particular acetates, enanthates, caproates, valerates and other pharmaceutically acceptable esters with C₂-C₁₀-alkanoyl radicals, which are mainly bonded to the hydroxyl group in position 17. Norethisterone acetate, i.e. 17β-hydroxy-19-nor-17α-pregn-4-en-20-yn-3-one acetate of the formula (Vg1)

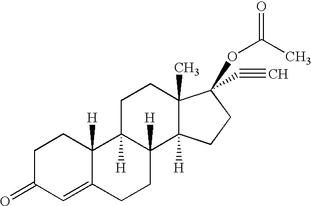

which is also referred to as 17α-ethynyl-19-nor-testosterone acetate, is particularly preferred.

Another norethisterone ester which should be mentioned is norethisterone enanthate.

Medicaments of the invention comprise an effective amount of norethisterone. Depending on the required dosage, the norethisterone content is ordinarily from 0.01 to 1.5% by weight and preferably 0.1 to 0.5% by weight. Norethisterone derivatives are added in equivalent amounts, for example about 0.186% by weight of norethisterone acetate (equivalent to 0.163% by weight of norethisterone).

A particularly advantageous embodiment of the present invention relates to medicaments in which the progestin, in particular norethisterone acetate, is present in dissolved form.

In a further particular embodiment, the present invention relates to medicaments with a combination of active ingredients which, besides norethisterone or norethisterone derivatives, comprise further active ingredients which are to be combined in particular with progestins.

In a preferred embodiment, medicaments of the invention comprise, besides norethisterone or norethisterone derivatives, at least one estrogen which can be administered transcutaneously, of which estradiol and estradiol derivatives should be mentioned in particular.

The term estradiol refers according to the invention to 3,17β-dihydroxy-Δ^{1,2,5,(10)}-estratriene of the formula (VI)

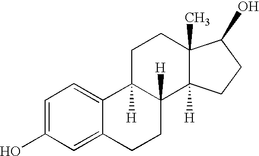

Estradiol is normally employed as anhydrate or as hemihydrate.

Estradiol derivatives which can be used according to the invention include, in particular, estradiol esters, e.g. esters of the formula (VIII)

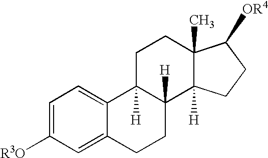

in which R³ and R⁴ are, independently of one another, an aliphatic or aromatic radical having 1 to 30 carbon atoms. Estradiol 3-benzoate, estradiol 17-cypionate, estradiol 3,17-dipropionate, estradiol 17-undecylate and estradiol 17-valerate should be mentioned here as examples.

Where present, medicaments of the invention comprise an amount, which is effective in combination with norethisterone, of an estrogen. The estrogen content is ordinarily from 0.01 to 1.5% by weight and preferably 0.02 to 0.5% by weight, for example about 0.06% by weight of estradiol. Estradiol derivatives are used in equivalent amounts.

A particularly preferred embodiment relates to a medicament of the invention based on norethisterone acetate and estradiol.

The medicaments of the invention are among the semisolid drug forms. Semisolid in the sense of the invention has the generally customary meaning indicated in the relevant monographs and pharmacopeias. The preparations are, in particular, capable of being spread, and they should be of spreadable consistency at room temperature, that is to say ordinarily about 20 to 25° C.

Spreadable dermatologicals of the invention may be divided in accordance with dermatological aspects into ointments, creams, gels and pastes, it being possible from the pharmaceutical viewpoint to use the term “ointment” for all spreadable preparations for dermal use, i.e. on the skin or mucosa.

The semisolid drug forms of the invention comprise a simple or composite base in which norethisterone or norethisterone derivatives are dissolved or dispersed. The bases may be formed from natural or synthetic substances, and they may be single phase or multiphase systems and have hydrophilic or hydrophobic (lipophilic) properties, depending on the nature of the base.

Ointments are, according to the invention, semisolid drug forms with a uniform base in which solid or liquid substances can be dissolved and/or dispersed.

Creams are, according to the invention, multiphase preparations consisting of a lipophilic and an aqueous phase.

Gels are based on gelated liquids which are obtainable with the aid of suitable swelling agents (gel formers).

Pastes are, according to the invention, semisolid preparations whose bases comprise considerable amount of finely dispersed powders.

Suitable bases can be formulated for example with hydrocarbons, in particular petrolatum, triglycerides, for example liquid fats such as peanut oil, olive oil, sunflower oil and castor oil, and spreadable fats such as pork fat, besides these natural fats also hydrogenated fats such as hydrogenated castor oil, and synthetic fats; polyethylene glycols (macrogols) with low to medium molecular weights, which ordinarily vary in the range from 200 to 5,000, for example polyethylene glycol 300, 400, 1,500 or 4,000 and, in particular, mixtures thereof; water-absorbing bases, also referred to as absorption bases, for example lipophilic water-absorbing bases such as wool wax, and hydrophilic water-absorbing bases such as emulsifying cetyl stearyl alcohol; gel formers, for example inorganic hydrogel formers or organic hydrogel formers; water and other hydrophilic solvents such as short-chain alcohols, e.g. ethanol.

In a preferred embodiment, semisolid medicaments of the invention are formulated as gel and, in particular, as hydrogel.

These comprise spreadable preparations with, in particular, a hydrophilic phase.

The hydrophilic phase is ordinarily formed from solvent, that is to say in particular from water, and, where appropriate, hydrophilic solvents, for example short-chain alcohols such as ethanol, or polyethylene glycols with low to medium molecular weight. The proportion of hydrophilic phase is ordinarily from 50 to 99% by weight and preferably 70 to 95% by weight, for example about 50% by weight of water and 40% of ethanol. The medicament preferably comprises the minimum amount of short-chain alcohols, in particular ethanol. A content of less than 50% by weight is advantageous.

Examples of suitable gel formers are:

a) inorganic gel formers such as

colloidal silica and mixtures thereof with alumina;

magnesium aluminum silicates, e.g. bentonites;

b) organic gel formers such as:

natural substances, in particular gelatin, polysaccharides (e.g. starch, pectin, tragacanth, alginates, xanthan gum);

semisynthetic gel formers, in particular cellulose ethers (e.g. methylcellulose, ethylcellulose, hydroxyethylcellulose, hydroxypropylcellulose, hydroxypropylmethylcellulose, sodium carboxymethylcellulose), starch derivatives, pectin derivatives;

fully synthetic gel formers such as polyvinyl alcohol, polyvinylpyrrolidones, ethylene oxide/propylene oxide block copolymers, carboxyvinyl polymers, and polymers and copolymers of acrylic and methacrylic acids.

Preference is given according to the invention to the acrylic acid polymers (CTFA name carbomer) normally used as gel formers, or the aforementioned cellulose derivatives.

The amount of gel former is normally such that the resulting gel has the desired rheological properties. It is accordingly possible for gels to have an elastic consistency (lyogel) or be plastically deformable. Ordinarily, from 0.1 to 10% by weight and preferably 0.5 to 5% by weight of gel former are used, for example about 1.4% by weight of carbomer.

In addition, the semisolid medicaments of the invention and, in particular, the gels comprise not only solvent but advantageously also one or more solubilizers. One function of suitable solubilizers may be to enhance percutaneous absorption of the active ingredients. Usable solubilizers may therefore be selected from the group of permeation enhancers. Examples which should be mentioned are monohydric or polyhydric alcohols such as benzyl alcohol, ethylene glycol, propylene glycol, glycerol and sorbitol, and ethers and esters thereof, for example diethylene glycol monoalkyl ethers preferably having 1 to 4 carbon atoms in the alkyl moiety, in particular diethylene glycol monoethyl ether, dimethylisosorbitol or glyceryl caprylate. Ethoxylated glycerides such as, for example, PEG 6 caprylic/capric acid glycerides are equally suitable. Emulsifiers may also be suitable, such as, for example, polysorbates, sorbitan fatty acid esters or polyethylene glycol 400 stearate. When these solubilizers are used, the content of short-chain alcohols can be chosen to be comparatively low.

Where present, medicaments of the invention ordinarily comprise from 0.5 to 20% by weight and preferably 1 to 10% by weight of solubilizer, for example about 5% by weight of diethylene glycol monoethyl ether.

The semisolid medicaments of the invention may additionally comprise further suitable additions such as neutralizing agents, for example triethanolamine, sodium hydroxide solution, tris buffer; preservatives; skin oils; skin-protecting substances; skincare agents.

In a preferred embodiment of the present invention, the medicaments comprise

0.01 to 1.5% by weight and, in particular, about 0.186% by weight of norethisterone acetate or a bioequivalent amount of a norethisterone derivative;

0.01 to 1.5% by weight and, in particular, about 0.06% by weight of estradiol or a bioequivalent amount of an estradiol derivative;

0.01 to 1.5% by weight and, in particular, about 0.2% by weight of ascorbic acid or a derivative and/or salt thereof in an equivalent amount in respect of the redox action.

It is advantageous within the scope of the embodiment described above for the medicaments to comprise:

0.01 to 1.0% by weight and, in particular, about 0.1% by weight of EDTA or another chelating agent in an equivalent amount in respect of the chelating action and/or

0.01 to 1.5% by weight and, in particular, about 0.1% by weight of lysine and/or fumaric acid or derivatives and/or salts thereof in an equivalent amount in respect of the redox action.

Hydrogels within the scope of the embodiment described above comprise:

0.1 to 10% by weight and, in particular, about 1.4% by weight of carbomer or another gel former in an equivalent amount in respect of the gel formation; and

50 to 99% by weight and, in particular, about 90% by weight of an aqueous ethanolic mixture or an equivalent amount of a hydrophilic solvent or mixture of solvents.

The hydrogels specified within the scope of the embodiment described above may advantageously comprise:

0.5 to 20% by weight and, in particular, about 5% by weight of diethylene glycol monoethyl ether or other solubilizers in an equivalent amount in respect of the solubilizing action; and/or

0.1 to 5% by weight and, in particular, about 1.6% by weight of triethanolamine or other neutralizing agents in an equivalent amount in respect of the basicity;

where the ethanol content can advantageously be less than 50% by weight and, in particular, less than 45% by weight.

The medicaments are produced in a manner known per se. The starting materials necessary for this are known from the literature.

The administration of a medicament of the invention ordinarily takes place by the semisolid preparation being applied and rubbed in, one or more times, in an amount equivalent to the desired dosage, on the skin or mucosa, for example of the arms, of the thighs or of the lower abdomen. The active ingredient concentration in the preparation is normally such that about 0.5 to 5 g of gel are to be applied each day.

Medicaments of the invention are used in particular in the area of hormone replacement therapy, that is to say in particular for the treatment of symptoms associated with a decline in estradiol production by the ovaries during and after the menopause or after ovarectomy (climacteric syndrome, for example hot flushes, night sweats, atrophic manifestations in the urogenital tract). The use can take place as part of a sequential or continuous therapy.

Unless otherwise indicated, data in % by weight are based on a total weight of the medicament (of the formulation).

The present invention is now illustrated by means of the following example:

### EXAMPLE 1

### Estradiol/norethisterone Acetate Gel

The following components were processed to a gel in a manner known per se:

| Component | Amount [g] |
|---|---|
| Estradiol 1/2 H₂O | 0.62 |
| Norethisterone acetate | 1.86 |
| EDTA 2Na | 1.00 |
| Ascorbic acid | 2.00 |
| Carbomer | 14.00 |
| Triethanolamine | 16.00 |
| Diethylene glycol | 50.00 |
| monoethyl ether | |
| Ethanol 96% | 417.00 |
| Purified water | 497.52 |
| | 1000.00 |

### EXAMPLE 2

### Stability Comparison

A gel A comparable with example 1 but additionally comprising α-tocopherol was subjected to a stability test (about 22 months at room temperature). The extent of decomposition of the active ingredients was determined and compared with a corresponding gel B which, although containing no ascorbic acid, did contain α-tocopherol and had been stored at room temperature for about 18 months:

| Decomposition products | Gel A | Gel B |
|---|---|---|
| 6α-, 6β-Hydroxynorethisterone acetate | 0.24% | 1.83% |
| 6-Ketonorethisterone acetate | 0.05% | 1.71% |
| Total | 0.90% | 6.37% |

It is clearly evident that the gel of the invention had considerably better stability of active ingredients than did the comparable gel which contained only α-tocopherol as antioxidant but no ascorbic acid.

We claim

1. A semisolid transcutaneous medicament based on at least one oxidation-sensitive progestin or a pharmaceutically acceptable derivative thereof, which comprises ascorbic acid, a pharmaceutically acceptable derivative and/or salt thereof.

2. A medicament as claimed in claim 1, wherein the ascorbic acid content is from 0.01 to 1.5% by weight.

3. A medicament as claimed in claim 1 or 2, which comprises at least one chelating agent.

4. A medicament as claimed in claim 3, wherein the chelating agent is EDTA or a pharmaceutically acceptable salt thereof.

5. A medicament as claimed in claim 3, wherein the chelating agent content is from 0.01 to 1% by weight.

6. A medicament as claimed in claim 1, which comprises at least one solubilizer.

7. A medicament as claimed in claim 6, wherein the solubilizer is a diethylene glycol mono-C₁₋₄-alkyl ether or dimethylisosorbitol.

8. A medicament as claimed in claim 6 or 7, wherein the ethanol content of the medicament is less than 50% by weight.

9. A medicament as claimed in claim 1, wherein the progestin has a basic steroid structure which is monounsaturated in the 4,5 position.

10. A medicament as claimed in claim 9, wherein the progestin is allylestrenol, desogestrel, gestodene, hydroxyprogesterone, lynestrenol, norethisterone, norgestrel, levonorgestrel or a pharmaceutically acceptable derivative thereof.

11. A medicament as claimed in claim 10, based on norethisterone acetate.

12. A medicament as claimed in claim 11, wherein the norethisterone content is from 0.01 to 1.5% by weight.

13. A medicament as claimed in claim 1, which comprises an estrogen which can be administered transcutaneously.

14. A medicament as claimed in claim 13, wherein the estrogen which can be administered transcutaneously is estradiol or a pharmaceutically acceptable derivative thereof.

15. A medicament as claimed in claim 13, wherein the estrogen content is from 0.01 to 1.5% by weight.

16. A medicament as claimed in claim 1 in the form of a gel.
